(19) United States
(12) Patent Application Publication
(10) Pub. No.: US 2002/0082498 A1
(43) Pub. Date: Jun. 27, 2002

(54) Intra-operative image-guided neurosurgery with augmented reality visualization

(75) Inventors: Michael Wendt, Hoboken, NJ (US); Ali R. Bani-Hashemi, Walnut Creek, CA (US); Frank Sauer, Princeton, NJ (US)

Correspondence Address: Siemens Corporation, Intellectual Property Department, 186 Wood Avenue South, Iselin, NJ, 08830, US

(73) Assignee: Siemens Corporate Research, Inc.

(21) Appl. No.: 09/971,554

(22) Filed: Oct. 5, 2001

Related U.S. Application Data
(60) Provisional application No. 60/238,253, filed on Oct. 5, 2000.
(60) Provisional application No. 60/279,931, filed on Mar. 29, 2001.

(51) Int. Cl. A61B 5/05

(52) U.S. Cl. 600/411; 600/414; 600/427; 600/426

(57) ABSTRACT

Apparatus for image-guided surgery includes medical imaging apparatus. The imaging apparatus is utilized for capturing 3-dimensional (3D) volume data of patient portions in reference to a coordinate system. A computer processes the volume data so as to provide a graphical representation of the data. A stereo camera assembly captures a stereoscopic video view of a scene including at least portions of the patient. A tracking system measures pose data of the stereoscopic video view in reference to the coordinate system. The computer is utilized for rendering the graphical representation and the stereoscopic video view in a blended way in conjunction with the pose data so as to provide a stereoscopic augmented image. A head-mounted video-see-through display displays the stereoscopic augmented image.

Reference is hereby made to Provisional Patent Application No. 60/238,253 entitled INTRA-OPERATIVE-MR GUIDED NEUROSURGERY WITH AUGEMENTED REALITY VISUALIZATION, filed Oct. 10, 2000 in the names of Wendt et al.; and to Provisional Patent Application No. 60/279,931 entitled METHOD AND APPARATUS FOR AUGMENTED REALITY VISUALIZATION, filed Mar. 29, 2001 in the name of Sauer, whereof the disclosures are hereby herein incorporated by reference.

The present invention relates to the field of image-guided surgery, and more particularly to MR-guided neurosurgery wherein imaging scans, such as magnetic resonance (MR) scans, are taken intra-operatively or inter-operatively.

In the practice of neurosurgery, an operating surgeon is generally required to look back and forth between the patient and a monitor displaying patient anatomical information for guidance in the operation. In this manner, a form of “mental mapping” occurs of the image information observed on the monitor and the brain.

Typically, in the case of surgery of a brain tumor, 3-dimensional (3D) volume images taken with MR (magnetic resonance) and CT (computed tomography) scanners are used for diagnosis and for surgical planning.

After opening of the skull (craniotomy), the brain, being non-rigid in its physical the brain will typically further deform. This brain shift makes the pre-operative 3D imaging data fit the actual brain geometry less and less accurately so that it is significantly out of correspondence with what is confronting the surgeon during the operation.

However, there are tumors that look like and are textured like normal healthy brain matter so that they are visually indistinguishable. Such tumors can be distinguished only by MR data and reliable resection is generally only possible with MR data that are updated during the course of the surgery. The term “intra-operative” MR imaging usually refers to MR scans that are being taken while the actual surgery is ongoing, whereas the term “inter-operative” MR imaging is used when the surgical procedure is halted for the acquisition of the scan and resumed afterwards.

Equipment has been developed by various companies for providing intra/inter-operative MR imaging capabilities in the operating room. For example, General Electric has built an MR scanner with a double-doughnut-shaped magnet, where the surgeon has access to the patient inside the scanner.

U.S. Pat. No. 5,740,802 entitled COMPUTER GRAPHIC AND LIVE VIDEO SYSTEM FOR ENHANCING VISUALIZATION OF BODY STRUCTURES DURING SURGERY, assigned to General Electric Company, issued Apr. 21, 1998 in the names of Nafis et al., is directed to an interactive surgery planning and display system which mixes live video of external surfaces of the patient with interactive computer generated models of internal anatomy obtained from medical diagnostic imaging data of the patient. The computer images and the live video are coordinated and displayed to a surgeon in real-time during surgery allowing the surgeon to view internal and external structures and the relation between them simultaneously, and adjust his surgery accordingly. In an alternative embodiment, a normal anatomical model is also displayed as a guide in reconstructive surgery. Another embodiment employs three-dimensional viewing.

Work relating to ultrasound imaging is disclosed by Andrei State, Mark A. Livingston, Gentaro Hirota, William F. Garrett, Mary C. Whitton, Henry Fuchs, and Etta D. Pisano, “Technologies for Augmented Reality Systems: realizing Ultrasound-Guided Needle Biopsies, “Proceed. of SIGGRAPH (New Orleans, La., Aug. 4-9, 1996), in Computer Graphics Proceedings, Annual Conference Series 1996, ACM SIGGRAPH, 439-446.

For inter-operative imaging, Siemens has built a combination of MR scanner and operating table where the operating table with the patient can be inserted into the scanner for MR image capture (imaging position) and be withdrawn into a position where the patient is accessible to the operating team, that is, into the operating position.

In the case of the Siemens equipment, the MR data are displayed on a computer monitor. A specialized neuroradiologist evaluates the images and discusses them with the neurosurgeon. The neurosurgeon has to understand the relevant image information and mentally map it onto the patient's brain. While such equipment provides a useful modality, this type of mental mapping is difficult and subjective and cannot preserve the complete accuracy of the information.

An object of the present invention is to generate an augmented view of the patient from the surgeon's own dynamic viewpoint and display the view to the surgeon.

The use of Augmented Reality visualization for medical applications has been proposed as early as 1992; see, for example, M. Bajura, H. Fuchs, and R. Ohbuchi. “Merging Virtual Objects with the Real World: Seeing Ultrasound Imagery within the Patient.” Proceedings of SIGGRAPH '92 (Chicago, Ill., Jul. 26-31, 1992). In Computer Graphics 26, #2 (July 1992): 203-210.

As herein used, the “augmented view” generally comprises the “real” view overlaid with additional “virtual” graphics. The real view is provided as video images. The virtual graphics is derived from a 3D volume imaging system. Hence, the virtual graphics also corresponds to real anatomical structures; however, views of these structures are available only as computer graphics renderings.

The real view of the external structures and the virtual view of the internal structures are blended with an appropriate degree of transparency, which may vary over the field of view. Registration between real and virtual views makes all structures in the augmented view appear in the correct location with respect to each other.

In accordance with an aspect of the invention, the MR data revealing internal anatomic structures are shown in-situ, overlaid on the surgeon's view of the patient. With this Augmented Reality type of visualization, the derived image of the internal anatomical structure is directly presented in the surgeon's workspace in a registered fashion.

In accordance with an aspect of the invention, the surgeon wears a head-mounted display and can examine the spatial relationship between the anatomical structures from varying positions in a natural way.

In accordance with an aspect of the invention, the need is practically eliminated for the surgeon to look back and forth between monitor and patient, and to mentally map the image information to the real brain. As a consequence, the surgeon can better focus on the surgical task at hand and perform the operation more precisely and confidently.

The invention will be more fully understood from the following detailed description of preferred embodiments, in conjunction with the Drawings, in which

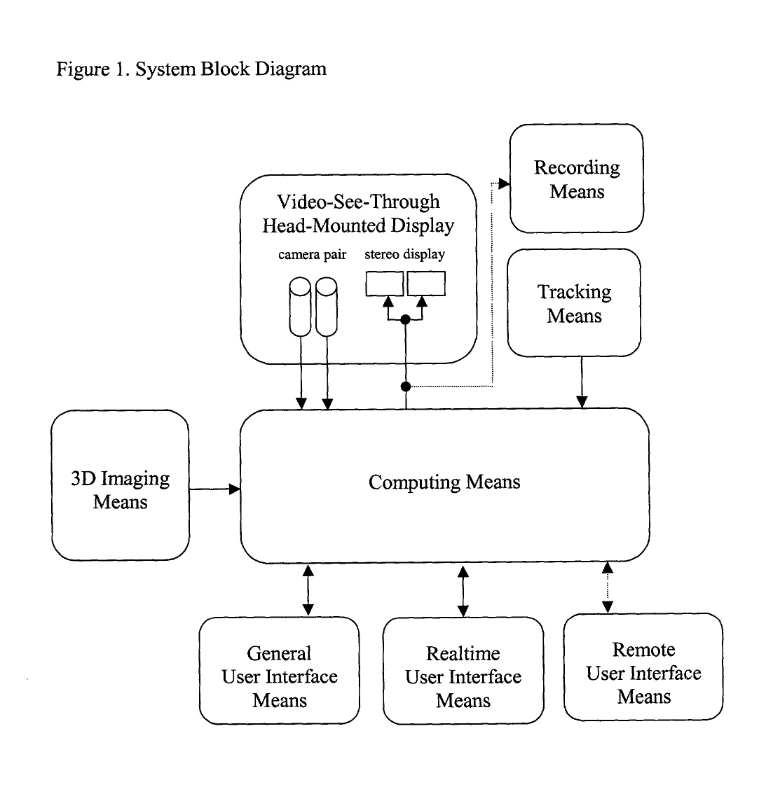

FIG. 1 shows a system block diagram in accordance with the invention;

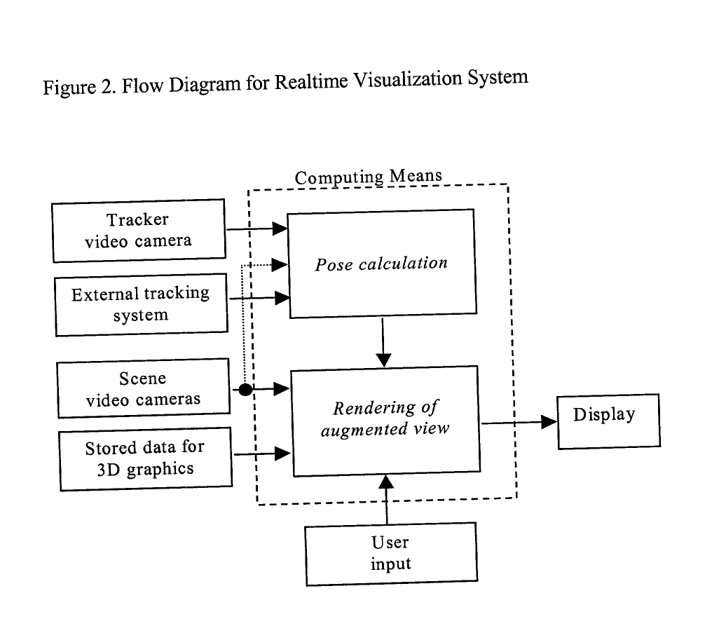

FIG. 2 shows a flow diagram in accordance with the invention;

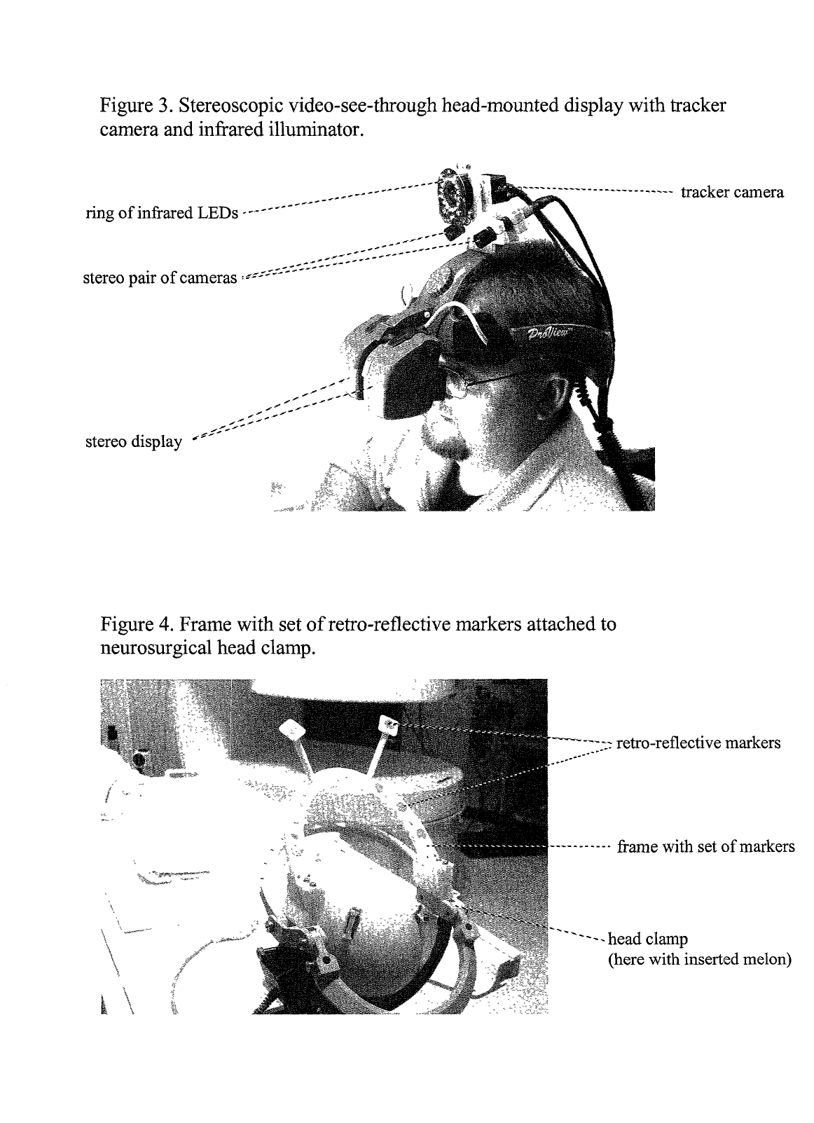

FIG. 3 shows a headmounted display as may be used in an embodiment of the invention;

FIG. 4 shows a frame in accordance with the invention;

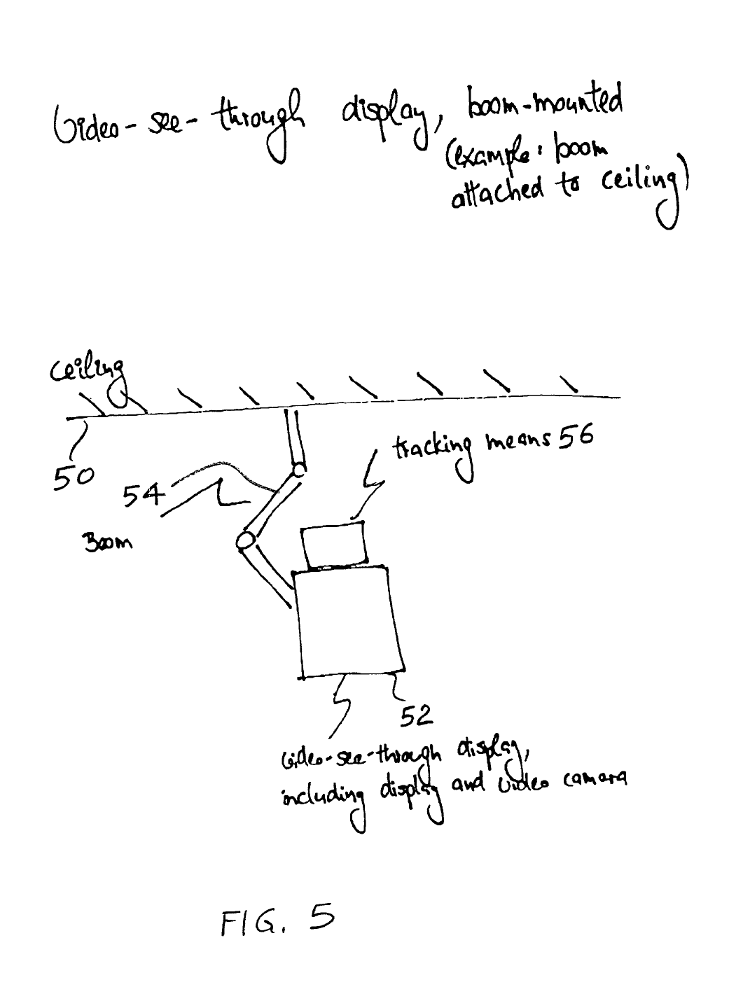

FIG. 5 show a boom-mounted see-through display in accordance with the invention;

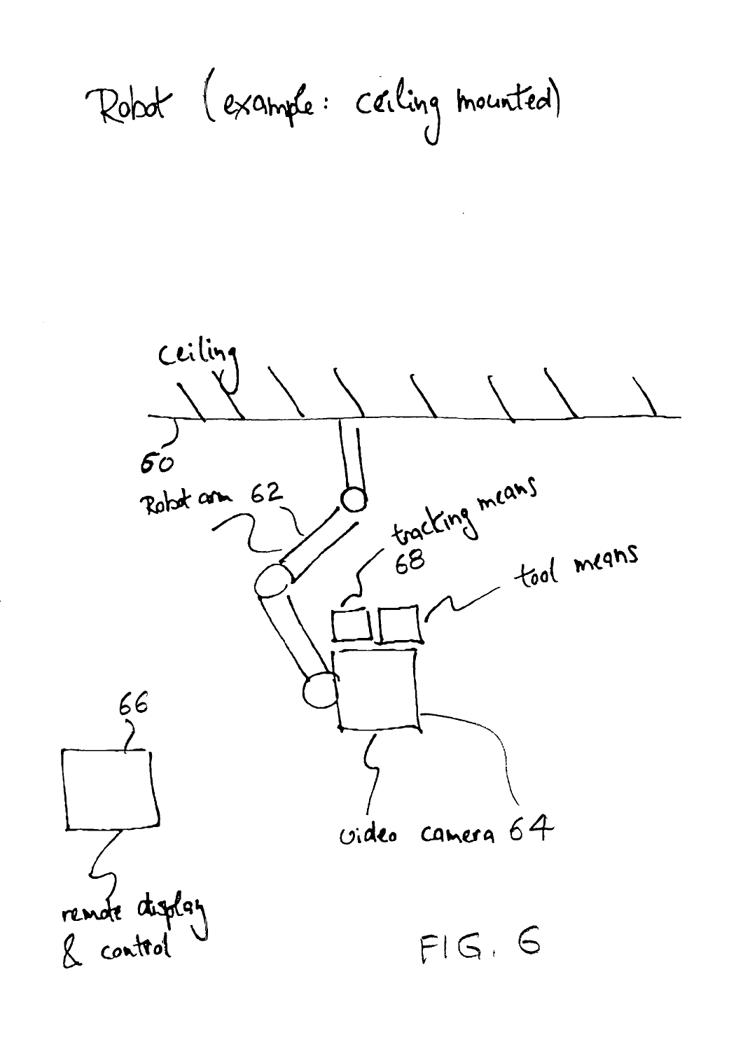

FIG. 6 shows a robotic arm in accordance with the invention;

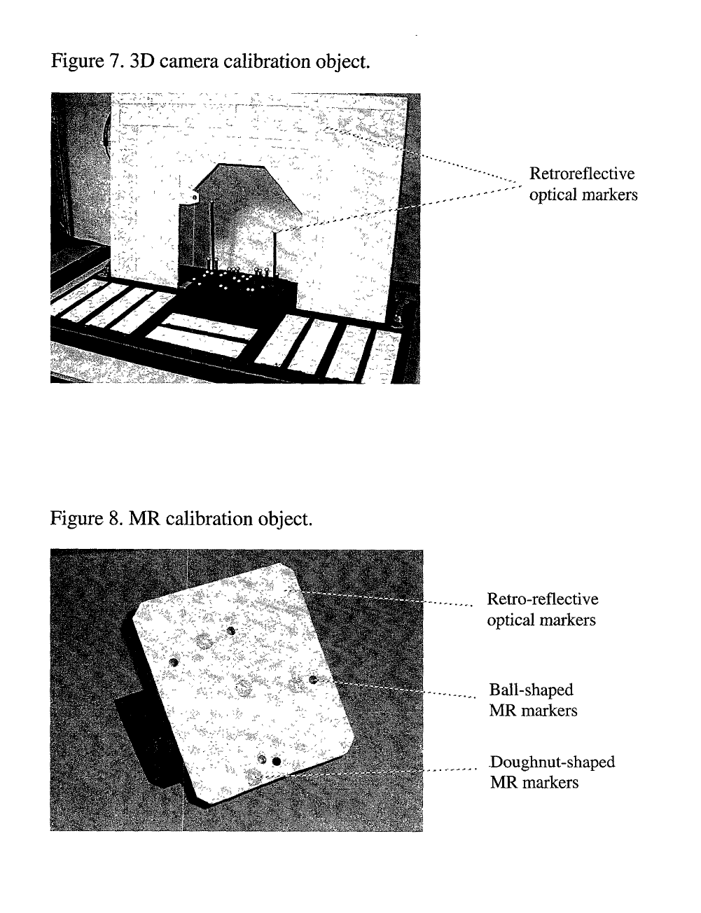

FIG. 7 shows a 3D camera calibration object as may be used in an embodiment of the invention; and

FIG. 8 shows an MR calibration object as may be used in an embodiment of the invention. Ball-shaped MR markers and doughnut shaped MR markers are shown

In accordance with the principles of the present invention, the MR information is utilized in an effective and optimal manner. In an exemplary embodiment, the surgeon wears a stereo video-see-through head-mounted display. A pair of video cameras attached to the head-mounted display captures a stereoscopic view of the real scene. The video images are blended together with the computer images of the internal anatomical structures and displayed on the head-mounted stereo display in real time. To the surgeon, the internal structures appear directly superimposed on and in the patient's brain. The surgeon is free to move his or her head around to view the spatial relationship of the structures from varying positions, whereupon a computer provides the precise, objective 3D registration between the computer images of the internal structures and the video images of the real brain. This in situ or “augmented reality” visualization gives the surgeon intuitively based, direct, and precise access to the image information in regard to the surgical task of removing the patient's tumor without hurting vital regions.

In an alternate embodiment, the stereoscopic video-see-through display may not be head-mounted but be attached to an articulated mechanical arm that is, e.g., suspended from the ceiling (reference to “videoscope” provisional filing)(include in claims). For our purpose, a video-see-through display is understood as a display with a video camera attachment, whereby the video camera looks into substantially the same direction as the user who views the display. A stereoscopic video-see-through display combines a stereoscopic display, e.g. a pair of miniature displays, and a stereoscopic camera system, e.g. a pair of cameras.

FIG. 1 shows the building blocks of an exemplary system in accordance with the invention.

A 3D imaging apparatus 2, in the present example an MR scanner, is used to capture 3D volume data of the patient. The volume data contain information about internal structures of the patient. A video-see-through head-mounted display 4 gives the surgeon a dynamic viewpoint. It comprises a pair of video cameras 6 to capture a stereoscopic view of the scene (external structures) and a pair of displays 8 to display the augmented view in a stereoscopic way.

A tracking device or apparatus 10 measures position and orientation (pose) of the pair of cameras with respect to the coordinate system in which the 3D data are described.

The computer 12 comprises a set of networked computers. One of the computer tasks is to process, with possible user interaction, the volume data and provide one or more graphical representations of the imaged structures: volume representations and/or surface representations (based on segmentation of the volume data). In this context, we understand the term graphical representation to mean a data set that is in a “graphical” format (e.g. VRML format), ready to be efficiently visualized respectively rendered into an image. The user can selectively enhance structures, color or annotate them, pick out relevant ones, include graphical objects as guides for the surgical procedure and so forth. This pre-processing can be done “off-line”, in preparation of the actual image guidance.

Another computer task is to render, in real time, the augmented stereo view to provide the image guidance for the surgeon. For that purpose, the computer receives the video images and the camera pose information, and makes use of the pre-processed 3D data, i.e. the stored graphical representation If the video images are not already in digital form, the computer digitizes them. Views of the 3D data are rendered according to the camera pose and blended with the corresponding video images. The augmented images are then output to the stereo display.

An optional recording means 14 allows one to record the augmented view for documentation and training. The recording means can be a digital storage device, or it can be a video recorder, if necessary, combined with a scan converter.

A general user interface 16 allows one to control the system in general, and in particular to interactively select the 3D data and pre-process them.

A realtime user interface 18 allows the user to control the system during its realtime operation, i.e. during the realtime display of the augmented view. It allows the user to interactively change the augmented view, e.g. invoke an optical or digital zoom, switch between different degrees of transparency for the blending of real and virtual graphics, show or turn off different graphical structures. A possible hands-free embodiment would be a voice controlled user interface.

An optional remote user interface 20 allows an additional user to see and interact with the augmented view during the system's realtime operation as described later in this document.

For registration, a common frame of reference is defined, that is, a common coordinate system, to be able to relate the 3D data and the 2D video images, with the respective pose and pre-determined internal parameters of the video cameras, to this common coordinate system.

The common coordinate system is most conveniently one in regard to which the patient's head does not move. The patient's head is fixed in a clamp during surgery and intermittent 3D imaging. Markers rigidly attached to this head clamp can serve as landmarks to define and locate the common coordinate system.

FIG. 4 shows as an example a photo of a head clamp 4-2 with an attached frame of markers 4-4. The individual markers are retro-reflective discs 4-6, made from 3M's Scotchlite 8710 Silver Transfer Film. A preferred embodiment of the marker set is in form of a bridge as seen in the photo. See FIG. 7.

The markers should be visible in the volume data or should have at least a known geometric relationship to other markers that are visible in the volume data. If necessary, this relationship can be determined in an initial calibration step. Then the volume data can be measured with regard to the common coordinate system, or the volume data can be transformed into this common coordinate system.

The calibration procedures follow in more detail. For correct registration between graphics and patient, the system needs to be calibrated. One needs to determine the transformation that maps the medical data onto the patient, and one needs to determine the internal parameters and relative poses of the video cameras to show the mapping correctly in the augmented view.

Camera calibration and camera-patient transformation. FIG. 7 shows a photo of an example of a calibration object that has been used for the calibration of a camera triplet consisting of a stereo pair of video cameras and an attached tracker camera. The markers 7-2 are retro-reflective discs. The 3D coordinates of the markers were measured with a commercial Optotrak® system. Then one can measure the 2D coordinates of the markers in the images, and calibrate the cameras based on 3D-2D point correspondences for example with Tsai's algorithm as described in Roger Y. Tsai, “A versatile Camera Calibration Technique for High-Accuracy 3D Machine Vision Metrology Using Off-the-Shelf TV Cameras and Lenses”, IEEE Journal of Robotics and Automation, Vol. RA-3, No. 4, August 1987, pages 323-344. For realtime tracking, one rigidly attaches a set of markers with known 3D coordinates to the patient (respectively a head clamp) defining the patient coordinate system. For more detailed information, refer to F. Sauer et al., “Augmented Workspace: Designing an AR Testbed,” IEEE and ACM Int. Symp. On Augmented Reality—ISAR 2000 (Munich, Germany, Oct. 5-6, 2000), pages 47-53.

MR data—patient transformation for the example of the Siemens inter-operative MR imaging arrangement. The patient's bed can be placed in the magnet's fringe field for the surgical procedure or swiveled into the magnet for MR scanning. The bed with the head clamp, and therefore also the patient's head, are reproducibly positioned in the magnet with a specified accuracy of ±1 mm. One can pre-determine the transformation between the MR volume set and the head clamp with a phantom and then re-apply the same transformation when mapping the MR data to the patient's head, with the head-clamp still in the same position.

FIG. 8 shows an example for a phantom that can be used for pre-determining the transformation. It consists of two sets of markers visible in the MR data set and a set of optical markers visible to the tracker camera. One type of MR markers is ball-shaped 8-2 and can, e.g., be obtained from Brainlab, Inc. The other type of MR markers 8-4 is doughnut-shaped, e.g. Multi-Modality Radiographics Markers from IZI Medical Products, Inc. In principle, only a single set of at least three MR markers is necessary. The disc-shaped retro-reflective optical markers 8-6 can be punched out from 3M's Scotchlite 8710 Silver Transfer Film. One tracks the optical markers, and—with the knowledge of the phantom's geometry—determines the 3D locations of the MR markers in the patient coordinate system. One also determines the 3D locations of the MR markers in the MR data set, and calculates the transformation between the two coordinate systems based on the 3D-3D point correspondences.

The pose (position and orientation) of the video cameras is then measured in reference to the common coordinate system. This is the task of the tracking means. In a preferred implementation, optical tracking is used due to its superior accuracy. A preferred implementation of optical tracking comprises rigidly attaching an additional video camera to the stereo pair of video cameras that provide the stereo view of the scene. This tracker video camera points in substantially the same direction as the other two video cameras. When the surgeon looks at the patient, the tracker video camera can see the aforementioned markers that locate the common coordinate system, and from the 2D locations of the markers in the tracker camera's image one can calculate the tracker camera's pose. As the video cameras are rigidly attached to each other, the poses of the other two cameras can be calculated from the tracker camera's pose, the relative camera poses having been determined in a prior calibration step. Such camera calibration is preferably based on 3D-2D point correspondences and is described, for example, in Roger Y. Tsai, “A versatile Camera Calibration Technique for High-Accuracy 3D Machine Vision Metrology Using Off-the-Shelf TV Cameras and Lenses”, IEEE Journal of Robotics and Automation, Vol. RA-3, No. 4, August 1987, pages 323-344.

FIG. 2 shows a flow diagram of the system when it operates in real-time mode, i.e. when it is displaying the augmented view in real time. The computing means 2-2 receives input from tracking systems, which are here separated into tracker camera (understood to be a head-mounted tracker camera) 2-4 and external tracking systems 2-6. The computing means perform pose calculations 2-8, based on this input and prior calibration data. The computing means also receives as input the real-time video of the scene cameras 2-10 and has available the stored data for the 3D graphics 2-12. In its graphics subsystem 2-14, the computing means renders graphics and video into a composite augmented view, according to the pose information. Via the user interface 2-16, the user can select between different augmentation modes (e.g. the user can vary the transparency of the virtual structures or select a digital zoom for the rendering process). The display 2-18 displays the rendered augmented view to the user.

To allow for a comfortable and relaxed posture of the surgeon during the use of the system, the two video cameras that provide the stereo view of the scene point downward at an angle, whereby the surgeon can work on the patient without having to bend the head down into an uncomfortable position. See the pending patent application Ser. No. ______ entitled AUGMENTED REALITY VISUALIZATION DEVICE, filed Sep. 17, 2001, Express Mail Label No. EL727968622US, in the names of Sauer and Bani-Hashemi, Attorney Docket No. 2001P14757US.

FIG. 3 shows a photo of a stereoscopic video-see-through head-mounted display. It includes the stereoscopic display 3-2 and a pair of downward tilted video cameras 3-4 for capturing the scene (scene cameras). Furthermore, it includes a tracker camera 3-6 and an infrared illuminator in form of a ring of infrared LEDs 3-8.

In another embodiment, the augmented view is recorded for documentation and/or for subsequent use in applications such as training.

It is contemplated that the augmented view can be provided for pre-operative planning for surgery.

In another embodiment, interactive annotation of the augmented view is provided to permit communication between a user of the head-mounted display and an observer or associate who watches the augmented view on a monitor, stereo monitor, or another head-mounted display so that the augmented view provided to the surgeon can be shared; for example, it can observed by neuroradiologist. The neuroradiologist can then point out, such as by way of an interface to the computer (mouse, 3D mouse, Trackball, etc.) certain features to the surgeon by adding extra graphics to the augmented view or highlighting existing graphics that is being displayed as part of the augmented view.

FIG. 5 shows a diagram of a boom-mounted video-see-through display. The video-see-through display comprises a display and a video camera, respectively a stereo display and a stereo pair of video cameras. In the example, the video-see-through display 52 is suspended from a ceiling 50 by a boom 54. For tracking, tracking means 56 are attached to the video-see-through display, more specifically to the video cameras as it is their pose that needs to be determined for rendering a correctly registered augmented view. Tracking means can include a tracking camera that works in conjunction with active or passive optical markers that are placed in the scene. Alternatively, tracking means can include passive or active optical markers that work in conjunction with an external tracker camera. Also, different kind of tracking systems can be employed such as magnetic tracking, inertial tracking, ultrasonic tracking, etc. Mechanical tracking is possible by fitting the joints of the boom with encoders. However, optical tracking is preferred because of its accuracy.

FIG. 6 shows elements of a system that employs a robotic arm 62, attached to a ceiling 60. The system includes a video camera respectively a stereo pair of video cameras 64. On a remote display and control station 66, the user sees an augmented video and controls the robot. The robot includes tools, e.g. a drill, that the user can position and activate remotely. Tracking means 68 enable the system to render an accurately augmented video view and to position the instruments correctly. Embodiments of the tracking means are the same as in the description of FIG. 5.

In an embodiment exhibiting remote use capability, a robot carries scene cameras. The tracking camera may then no longer be required as robot arm can be mechanically tracked. However, in order to establish the relationship between the robot and patient coordinate systems, the tracking camera can still be useful.

The user, sited in a remote location, can move the robot “head” around by remote control to gain appropriate views, look at the augmented views on a head-mounted display or other stereo viewing display or external monitor, preferably in stereo, to diagnose and consult. The remote user may also be able to perform actual surgery via remote control of the robot, with or without help of personnel present at the patient site.

In another embodiment in accordance with the invention, a video-see-through head-mounted display has downward looking scene camera/cameras. The scene cameras are video cameras that provide a view of the scene, mono or stereo, allowing a comfortable work position. The downward angle of the camera /cameras is such that—in the preferred work posture—the head does not have to be tilted up or down to any substantial degree.

In another embodiment in accordance with the invention, a video-see-through display comprises an integrated tracker camera whereby the tracker camera is forward looking or is looking into substantially the same direction as the scene cameras, tracking landmarks that are positioned on or around the object of interest. The tracker camera can have a larger field of view than the scene cameras, and can work in limited wavelength range (for example, the infrared wavelength range). See the afore-mentioned pending patent application Ser. No. ______ entitled AUGMENTED REALITY VISUALIZATION DEVICE, filed Sep. 17, 2001, Express Mail Label No. EL727968622US, in the names of Sauer and Bani-Hashemi, Attorney Docket No. 2001P14757US, hereby incorporated herein by reference.

In accordance with another embodiment of the invention wherein retroreflective markers are used, a light source for illumination is placed close to or around the tracker camera lens. The wavelength of the light source is adapted to the wavelength range for which the tracker camera is sensitive. Alternatively, active markers, for example small lightsources such as LEDs can be utilized as markers.

Tracking systems with large cameras that work with retroreflective markers or active markers are commercially available.

In accordance with another embodiment of the invention, a video-see-through display includes a digital zoom feature. The user can zoom in to see a magnified augmented view, interacting with the computer by voice or other interface, or telling an assistant to interact with the computer via keyboard or mouse or other interface.

It will be apparent that the present inventions provide certain useful characteristics and features in comparison with prior systems. For example, in reference to the system disclosed in the afore-mentioned U.S. Pat. No. 5,740,802, video cameras are attached to head-mounted display in accordance with the present invention, thereby exhibiting a dynamic viewpoint, in contrast with prior systems which provide a viewpoint, implicitly static or quasi-static, which is only “substantially” the same as the surgeon's viewpoint.

In contrast with a system which merely displays a live video of external surfaces of a patient and an augmented view to allow a surgeon to locate internal structures relative to visible external surfaces, the present invention makes it unnecessary for the surgeon to look at an augmented view, then determine the relative positions of external and internal structures and thereafter orient himself based on the external structures, drawing upon his memory of the relative position of the internal structures.

The use of a “video-see-through” head mounted display in accordance with the present invention provides an augmented view in a more direct and intuitive way without the need for the user to look back and forth between monitor and patient. This also results in better spatial perception because of kinetic (parallax) depth cues and there is no need for the physician to orient himself with respect to surface landmarks, since he is directly guided by the augmented view.

In such a prior art system mixing is performed in the video domain wherein the graphics is converted into video format and then mixed with the live video such that the mixer arrangement creates a composite image with a movable window which is in a region in the composite image that shows predominantly the video image or the computer image. In contrast, an embodiment in accordance with the present invention does not require a movable window; however, such a movable window may be helpful in certain kinds of augmented views. In accordance with a principle of the present invention, a composite image is created in the computer graphics domain whereby the live video is converted into a digital representation in the computer and therein blended together with the graphics.

Furthermore, in such a prior art system, internal structures are segmented and visualized as surface models; in accordance with the present invention, 3D images can be shown in surface or in volume representations.

The present invention has been described by way of exemplary embodiments. It will be understood by one of skill in the art to which it pertains that various changes, substitutions and the like may be made without departing from the spirit of the invention. Such changes are contemplated to be within the scope of the claims following.

What is claimed is:

1. A method for image-guided surgery comprising:
capturing 3-dimensional (3D) volume data of at least a portion of a patient;
processing said volume data so as to provide a graphical representation of said data;
capturing a stereoscopic video view of a scene including said at least a portion of said patient;
rendering said graphical representation and said stereoscopic video view in a blended manner so as to provide a stereoscopic augmented image; and
displaying said stereoscopic augmented image in a video-see-through display.

2. A method for image-guided surgery comprising:
capturing 3-dimensional (3D) volume data of at least a portion of a patient in reference to a coordinate system;
processing said volume data so as to provide a graphical representation of said data;
capturing a stereoscopic video view of a scene including said at least a portion of said patient;
measuring pose data of said stereoscopic video view in reference to said coordinate system;
rendering said graphical representation and said stereoscopic video view in a blended manner in conjunction with said pose data so as to provide a stereoscopic augmented image; and
displaying said stereoscopic augmented image in a video-see-through display.

3. A method for image-guided surgery in accordance with claim 1, wherein said step of capturing 3-dimensional (3D) volume data comprises obtaining magnetic-resonance imaging data.

4. A method for image-guided surgery in accordance with claim 1, wherein said step of processing said volume data comprises processing said data in a programmable computer.

5. A method for image-guided surgery in accordance with claim 1, wherein said step of capturing a stereoscopic video view comprises capturing a stereoscopic view by a pair of stereo cameras.

6. A method for image-guided surgery in accordance with claim 2, wherein said step of measuring pose data comprises measuring position and orientation of said pair of stereo cameras by way of a tracking device.

7. A method for image-guided surgery in accordance with claim 1, wherein said step of rendering said graphical representation and said stereoscopic video view manner in conjunction with said pose data comprises utilizing video images, and where necessary, digitizing said video images, said camera pose information, and stored volume data captured in a previous step for providing said stereoscopic augmented image.

8. A method for image-guided surgery in accordance with claim 1, wherein said step of displaying said stereoscopic augmented image in a video-see-through display comprises displaying said stereoscopic augmented image in a head-mounted video-see-through display.

9. Apparatus for image-guided surgery comprising:
means for capturing 3-dimensional (3D) volume data of at least a portion of a patient;
means for processing said volume data so as to provide a graphical representation of said data;
means for capturing a stereoscopic video view of a scene including said at least a portion of said patient;
means for rendering said graphical representation and said stereoscopic video view in a blended manner way so as to provide a stereoscopic augmented image; and
means for displaying said stereoscopic augmented image in a video-see-through display.

10. Apparatus for image-guided surgery comprising:
means for capturing 3-dimensional (3D) volume data of at least a portion of a patient in reference to a coordinate system;
means for processing said volume data so as to provide a graphical representation of said data;
means for capturing a stereoscopic video view of a scene including said at least a portion of said patient;
means for measuring pose data of said stereoscopic video view in reference to said coordinate system;
means for rendering said graphical representation and said stereoscopic video view in a blended manner in conjunction with said pose data so as to provide a stereoscopic augmented image; and
means for displaying said stereoscopic augmented image in a video-see-through display.

11. Apparatus for image-guided surgery in accordance with claim 9, wherein said means for capturing 3-dimensional (3D) volume data comprises means for obtaining magnetic-resonance imaging data.

12. Apparatus for image-guided surgery in accordance with claim 9, wherein said means for processing said volume data comprises means for processing said data in a programmable computer.

13. Apparatus for image-guided surgery in accordance with claim 9, wherein said means for capturing a stereoscopic video view comprises means for capturing a stereoscopic view by a pair of stereo cameras.

14. Apparatus for image-guided surgery in accordance with claim 9, wherein said means for measuring pose data comprises means for measuring position and orientation of said pair of stereo cameras by way of a tracking device.

15. Apparatus image-guided surgery in accordance with claim 9, wherein said means for rendering said graphical representation and said stereoscopic video view in a blended manner in conjunction with said pose data comprises means for utilizing video images, and where necessary, digitizing said video images, said camera pose information, and stored previously captured volume data captured for providing said stereoscopic augmented image.

16. Apparatus for image-guided surgery in accordance with claim 9, wherein said means for displaying said stereoscopic augmented image in a video-see-through display comprises a head-mounted video-see-through display.

17. Apparatus for image-guided surgery in accordance with claim 9, including a set of markers in predetermined relationship to said patient for defining said coordinate system.

18. Apparatus for image-guided surgery in accordance with claim 17, wherein said markers are identifiable in said volume data.

19. Apparatus for image-guided surgery in accordance with claim 18, wherein said means for displaying said stereoscopic augmented image in a video-see-through display comprises a boom-mounted video-see-through display.

20. Apparatus for image-guided surgery comprising:
medical imaging apparatus, said imaging apparatus being utilized for capturing 3-dimensional (3D) volume data of at least patient portions in reference to a coordinate system;
a computer for processing said volume data so as to provide a graphical representation of said data;
a stereo camera assembly for capturing a stereoscopic video view of a scene including said at least patient portions;
a tracking system for measuring pose data of said stereoscopic video view in reference to said coordinate system;
said computer being utilized for rendering said graphical representation and said stereoscopic video view in a blended way in conjunction with said pose data so as to provide a stereoscopic augmented image; and
a head-mounted video-see-through display for displaying said stereoscopic augmented image.

21. Apparatus for image-guided surgery in accordance with claim 20, wherein said medical imaging apparatus is one of X-ray computed tomography apparatus, magnetic resonance imaging apparatus, and 3D ultrasound imaging apparatus.

22. Apparatus for image-guided surgery in accordance with claim 20, wherein said coordinate system is defined in relation to said patient.

23. Apparatus for image-guided surgery in accordance with claim 22, including markers in predetermined relationship to said patient.

24. Apparatus for image-guided surgery in accordance with claim 23, wherein said markers are identifiable in said volume data.

25. Apparatus for image-guided surgery in accordance with claim 20, wherein said computer comprises a set of networked computers.

26. Apparatus for image-guided surgery in accordance with claim 25, wherein said computer processes said volume data with optional user interaction, and provides at least one graphical representation of said patient portions, said graphical representation comprising at least one of volume representations and surface representations based on segmentation of said volume data.

27. Apparatus for image-guided surgery in accordance with claim 26, wherein said optional user interaction allows a user to, in any desired combination, selectively enhance, color, annotate, single out, and identify for guidance in surgical procedures, at least a portion of said patient portions.

28. Apparatus for image-guided surgery in accordance with claim 20, wherein said tracking system comprises an optical tracker.

29. Apparatus for image-guided surgery in accordance with claim 20, wherein said stereo camera assembly are adapted for operating in an angled swiveled orientation, including a downward-looking orientation for allowing a user to operate without having to tilt the head downward.

30. Apparatus for image-guided surgery in accordance with claim 28, wherein said optical tracker comprises a tracker video camera in predetermined coupled relationship with said stereo camera assembly.

31. Apparatus for image-guided surgery in accordance with claim 28, wherein said optical tracker comprises a tracker video camera faces in substantially the same direction as said stereo camera assembly for tracking landmarks around the center area of view of said stereo camera assembly.

32. Apparatus for image-guided surgery in accordance with claim 31, wherein said tracker video camera exhibits a larger field of view than said stereo camera assembly.

33. Apparatus for image-guided surgery in accordance with claim 31, wherein said landmarks comprise optical markers.

34. Apparatus for image-guided surgery in accordance with claim 31, wherein said landmarks comprise reflective markers.

35. Apparatus for image-guided surgery in accordance with claim 34, wherein said reflective markers are illuminated by light of a wavelength suitable for said tracker video camera.

36. Apparatus for image-guided surgery in accordance with claim 20, wherein said video-see-through display comprises a zoom feature.

37. Apparatus for image-guided surgery in accordance with claim 31, wherein said landmarks comprise light-emitting markers.

38. Apparatus for image-guided surgery in accordance with claim 20, wherein said augmented view can be any combination: stored, replayed, remotely viewed, and simultaneously replicated for at least one additional user.

39. Apparatus for image-guided surgery comprising:
medical imaging apparatus, said imaging apparatus being utilized for capturing 3-dimensional (3D) volume data of at least patient portions in reference to a coordinate system;
a computer for processing said volume data so as to provide a graphical representation of said data;
a robot arm manipulator operable by user from a remote location;
a stereo camera assembly mounted on said robot arm manipulator for capturing a stereoscopic video view of a scene including said patient;
a tracking system for measuring pose data of said stereoscopic video view in reference to said coordinate system;
said computer being utilized for rendering said graphical representation and said stereoscopic video view in a blended way in conjunction with said pose data so as to provide a stereoscopic augmented image; and
a head-mounted video-see-through display for displaying said stereoscopic augmented image at said remote location.

40. Apparatus for image-guided surgery in accordance with claim 39, wherein said optical tracker comprises a tracker video camera in predetermined coupled relationship with said robot arm manipulator.

41. A method for image-guided surgery utilizing captured 3-dimensional (3D) volume data of at least a portion of a patient, said method comprising:
processing said volume data so as to provide a graphical representation of said data;
capturing a stereoscopic video view of a scene including said at least a portion of said patient;
rendering said graphical representation and said stereoscopic video view in a blended manner so as to provide a stereoscopic augmented image; and
displaying said stereoscopic augmented image in a video-see-through display.

42. A method for image-guided surgery utilizing 3-dimensional (3D) volume data of at least a portion of a patient, said data having been captured in reference to a coordinate system, said method comprising:
capturing 3-dimensional (3D) volume data of at least a portion of a patient processing said volume data so as to provide a graphical representation of said data;
capturing a stereoscopic video view of a scene including said at least a portion of said patient;
measuring pose data of said stereoscopic video view in reference to said coordinate system;
rendering said graphical representation and said stereoscopic video view in a blended manner in conjunction with said pose data so as to provide a stereoscopic augmented image; and
displaying said stereoscopic augmented image in a video-see-through display.

43. A method for image-guided surgery in accordance with claim 42, wherein said 3-dimensional (3D) volume data comprises magnetic-resonance imaging data.

44. A method for image-guided surgery in accordance with claim 42, wherein said step of processing said volume data comprises processing said data in a programmable computer.

45. A method for image-guided surgery in accordance with claim 42, wherein said step of capturing a stereoscopic video view comprises capturing a stereoscopic view by a pair of stereo cameras.

46. A method for image-guided surgery in accordance with claim 42, wherein said step of measuring pose data comprises measuring position and orientation of said pair of stereo cameras by way of a tracking device.

47. A method for image-guided surgery in accordance with claim 42, wherein said step of rendering said graphical representation and said stereoscopic video view in a blended way in conjunction with said pose data comprises utilizing video images, and where necessary, digitizing said video images, said camera pose information, and stored volume data captured in a previous step for providing said stereoscopic augmented image.

48. A method for image-guided surgery in accordance with claim 42, wherein said step of displaying said stereoscopic augmented image in a video-see-through display comprises displaying said stereoscopic augmented image in a head-mounted video-see-through display.

49. Apparatus for image-guided surgery utilizing captured 3-dimensional (3D) volume data of at least a portion of a patient, said apparatus comprising:
means for processing said volume data so as to provide a graphical representation of said data;
means for capturing a stereoscopic video view of a scene including said at least a portion of said patient;
means for rendering said graphical representation and said stereoscopic video view in a blended manner so as to provide a stereoscopic augmented image; and
means for displaying said stereoscopic augmented image in a video-see-through display.

50. Apparatus for image-guided surgery utilizing 3-dimensional (3D) volume data of at least a portion of a patient, said data having been captured in reference to a coordinate system, said apparatus comprising:
means for processing said volume data so as to provide a graphical representation of said data;
means for capturing a stereoscopic video view of a scene including said at least a portion of said patient;
means for measuring pose data of said stereoscopic video view in reference to said coordinate system;
means for rendering said graphical representation and said stereoscopic video view in a blended manner in conjunction with said pose data so as to provide a stereoscopic augmented image; and
means for displaying said stereoscopic augmented image in a video-see-through display.
